(19) United States
(12) Patent Application Publication
(10) Pub. No.: US 2020/0123122 A1
(43) Pub. Date: Apr. 23, 2020

(54) PRODUCTION AND USE OF FURAN COMPOUNDS

(71) Applicant: Coöperatie Koninklijke Cosun U.A., Breda (NL)

(72) Inventors: Luiz A. Canalle, Houten (NL); Jack A. M. van Schijndel, Moerdijk (NL); Dennis Molendijk, Dordrecht (NL); Robert Lazeroms, Sprundel (NL); Adeline Ranoux, Dordrecht (NL); Harry Raaijmakers, Breda (NL)

(73) Assignee: Coöperatie Koninklijke Cosun U.A., Breda (NL)

(21) Appl. No.: 16/625,857

(22) PCT Filed: Jun. 22, 2018
(86) PCT No.: PCT/NL2018/050404

§ 371 Date: Dec. 23, 2019

(30) Foreign Application Priority Data
Jun. 23, 2017 (EP) 17177604.0

(51) Int. Cl. C07D 307/54

(52) U.S. Cl. CPC C07D 307/54

(57) ABSTRACT

The present invention, relates to processes for the production and conversion of furan compounds. In certain embodiments the invention concerns the conversion of furfural compounds into a so-called Knoevenagel products and for the further conversion of such Knoevenagel products into furan commodities. The reactions involved, generally speaking, use benign chemicals and conditions and have good yields and selectivity, and it is expected that they can be implemented on a large (industrial) scale in an economically feasible manner. Also, with the processes developed by the present inventors, various highly interesting new furan commodities become available. The invention also provides various intermediate and end-products obtained with the processes of the invention.

### FIELD OF THE INVENTION

The present invention, generally stated, relates to processes for the production and conversion of furan compounds. In certain embodiments the invention concerns the conversion of furfural compounds into so-called Knoevenagel products and for the further conversion of such Knoevenagel products into said furan commodities. The invention also provides various intermediate and end-products obtained with the processes of the invention.

### BACKGROUND OF THE INVENTION

Society currently depends on petroleum for products such as gasoline, plastics, solvents, asphalt and jet fuel. At present, about 10-15% of petroleum is used for the production of high-value chemicals. However, petroleum is becoming increasingly expensive and scarce. Additionally, the environmental impact of using petroleum have become increasingly clear. Therefore, it is necessary that alternative, renewable carbon sources are developed to at least augment petroleum-sourced chemicals. Biomass constitutes a unique and promising candidate feedstock, containing sugars that can be converted into valuable chemical intermediates. The challenge in this respect is to find a cost-effective way of processing biomass, to yield chemical intermediates that are suitable for use with the existing technologies based on petroleum derived chemicals, ideally with no or little modification. In general, the concept of producing ‘platform’ chemical intermediates that can be subsequently converted into a diverse range of products is particularly interesting.

One major biomass-based strategy, generally stated, involves the depolymerization of polysaccharides to produce the monomers for selective conversion into such versatile platform chemicals. Such strategies will inherently involve intermediate materials with extensive and diverse functionality derived from sugars, which have excess oxygen, in contrast to the petroleum-based strategies where unfunctionalized alkanes are the primary starting material. Thus, in general, biomass based strategies will involve selective removal of excess functionality to produce molecules similar to those derived from petroleum. In each case the economic feasibility of producing chemicals from biomass will depend on the selectivities of the consecutive (catalytic) conversion strategies.

HMF (5-(hydroxymethyl)-2-furaldehyde), resulting from the acid catalysed dehydration of fructose, constitutes one of the key intermediates in many biomass-based strategies. It has been proposed that HMF could be utilized as an intermediate in the production of a wide range of products such as polymers, solvents, surfactants, pharmaceuticals, and plant protection agents. Despite this generally recognized potential of HMF, an economically viable method for large-scale production is still lacking. One of the big problems is that HMF is not very stable at the reaction conditions required for its formation and purification. With technologies available to date, it is nonetheless possible to produce reasonable quantities of HMF in a highly pure (crystalline) form and a commercial supplier has recently started production of such HMF products. However these HMF products are way too costly to really serve as a starting material for the commercial scale production of furan commodities. As an alternative, the unpurified or partly purified aqueous HMF solutions can be sourced against much lower costs. However, the presence of water and by-products from the fructose to HMF conversion in these solutions pose currently unmet challenges in the reactions for converting the HMF into furan commodities.

Reactions for the conversion of HMF into interesting furan commodities typically involve reactions where carbon atoms are added and/or oxygen is removed. Although many relevant reactions are well known to those skilled in the art, a serious challenge still resides in the design of highly selective conversion strategies that are efficient and economically feasible. Such strategies should, in particular also match the objective of reducing the environmental burden and should not involve the use of harmful chemicals and/or require high energy input. This is all the more a challenge, if the reactions were to start from the relatively inexpensive aqueous HMF solutions instead of the purified (crystalline) form.

It is generally understood that the development of new reaction pathways might open up possibilities to produce new commodities with very interesting properties.

It is the objective of the present invention to provide solutions to the challenges and objectives described above.

### SUMMARY OF THE INVENTION

The present inventors have developed new processes for the conversion of furfural compounds, such as HMF, into various furan commodities.

The reactions involved, generally speaking, use benign chemicals and conditions and have good yields and selectivity, and it is expected that they can be implemented on a large (industrial) scale in an economically feasible manner. Also, with the processes developed by the present inventors, various highly interesting furan commodities become available.

A first aspect of the invention presented herein resides in the conversion of furfural compounds, such as HMF, into a so-called Knoevenagel product, and forming a furan building block by subjecting the Knoevenagel product to a further reaction as for example a hydrogenation reaction, a decarboxylation reaction or a de-esterification reaction.

An exemplary embodiment of the process comprising hydrogenation of the Knoevenagel product is depicted in the below reaction scheme.

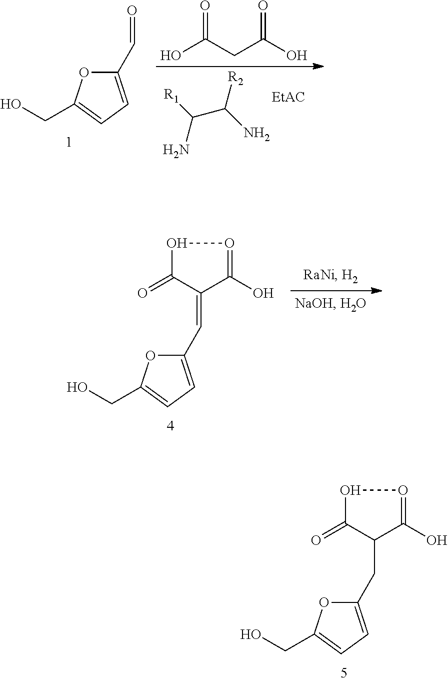

An exemplary embodiment of the process comprising decarboxylation of the Knoevenagel product is depicted in the below reaction scheme.

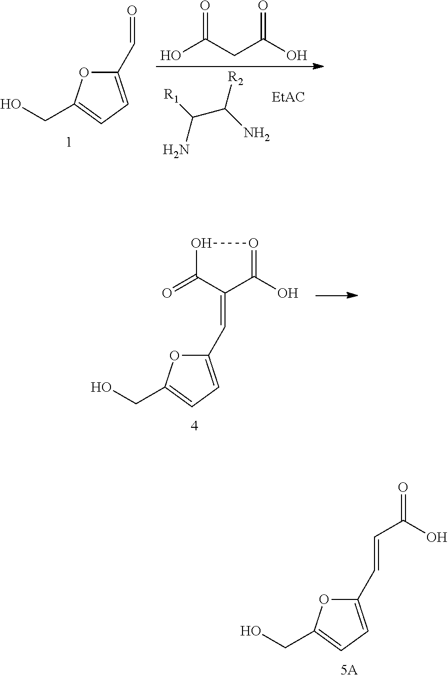

An exemplary embodiment of the process comprising a de-esterification, in particular a mono de-esterification of the Knoevenagel product is depicted in the below reaction scheme.

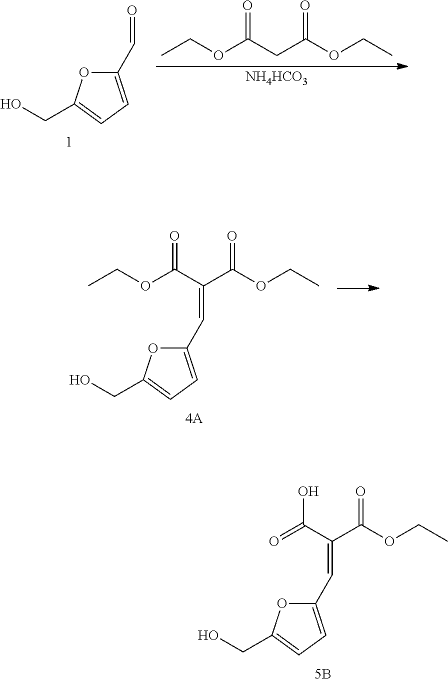

In the process of producing the furan compound (5, 5A, 5B) from the furfural compound (1), reactants and conditions can be applied which are relatively favorable from the environmental perspective. In addition, the present inventors have surprisingly found that the conversion of the Knoevenagel product (4, 4A) into the furan compound (5, 5A, 5B) proceeds at a high rate and with high selectivity, even when performed under relatively mild conditions.

Typically, in accordance with the invention, the Knoevenagel reaction comprises reacting the furfural compound with activated methylene compound, as will be explained in more detail herein, such as malonic acid and/or malonic acid esters. The use of malonic acid esters, such as diethyl malonate or dimethyl malonate, entails the particular advantage of relatively low cost/high availability.

Knoevenagel reactions of (biomass derived) furfural compounds with activated methylene compounds have been described in prior art document U.S. Pat. No. 8,236,972. This document, generally stated, concerns the production of fuels from renewable feedstocks. U.S. Pat. No. 8,236,972 teaches to subject the Knoevenagel product to a hydrodeoxygenation. Hydrodeoxygenation is a process resulting in the removal of oxygen atoms, which may be accomplished by reacting the Knoevenagel product with hydrogen or another reducing agent, in the presence of a suitable catalyst. U.S. Pat. No. 8,236,972 specifically advocates to remove at least 50% up to substantially all of the oxygen atoms, as is also illustrated by the reaction scheme presented as FIG. 1. The hydrodeoxygenated products, according to U.S. Pat. No. 8,236,972, comprise mixtures of alkanes, alkenes oxygenates or mixtures thereof with enhanced viscosity, energy density and/or stability, and are said to be useful in or as fuels.

The present invention thus differs substantially from the processes disclosed in U.S. Pat. No. 8,236,972 in that it provides highly selective reactions of the Knoevenagel product, wherein carboxylic acid functionality remains intact, rather than being hydrodeoxygenated. To the best knowledge of the present inventors, there has never been any prior teaching or disclosure of the conversion of furfural compounds into Knoevenagel products followed by hydrogenation, decarboxylation or de-esterification to produce furan building blocks comprising at least one intact carboxylic acid functionality selected from the group consisting of —COOH, —C(═O)—O—CH₃ and —C(═O)—O—CH₂CH₃.

A particular advantage of the process of the invention resides in the fact that it can be performed, not only with highly pure (crystalline) furfural compound, but also, with good efficiency, with the non- or partially purified aqueous solutions of the furfural compound, which are far less costly to produce/acquire. A further advantage of the present process is that it can be carried out mostly or even exclusively using chemicals that meet international green chemistry standards.

The inventors also established that it is particularly advantageous to perform the conversion of the furfural compound into the Knoevenagel product using ethyl acetate as the solvent. The present inventors established that certain Knoevenagel products obtained in accordance with the invention precipitate from ethyl acetate in extremely high purity. Hence, in accordance with these embodiments of the invention, a highly pure Knoevenagel product can be collected using a simple liquid-solid-separation technique, such as filtration. The Knoevenagel product thus obtained is suitable as a starting material in the further reactions disclosed herein, without any further purification steps.

Further aspects of the invention presented herein concern processes for the conversion of the furan compound. Preferred embodiments entail conversions by decarboxylation and esterification, yielding new furan commodities with highly interesting properties. An exemplary embodiment of the pathway is depicted in the below reaction scheme.

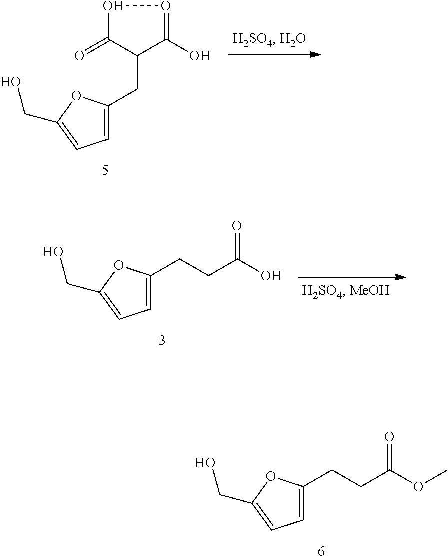

The invention also in particular pertains to a process of producing furan commodities, such as those represented by (3) and (6), from a furfural compound via the Knoevenagel reaction, followed by hydrogenation and subsequent decarboxylation and, optionally, esterification, in accordance with the foregoing. This overall synthetic route is remarkably efficient.

Furthermore the invention pertains to a process of producing furan commodities, such as those represented by (7), from a furfural compound via the Knoevenagel reaction, followed by de-esterification, in particular mono de-esterification and subsequent decarboxylation.

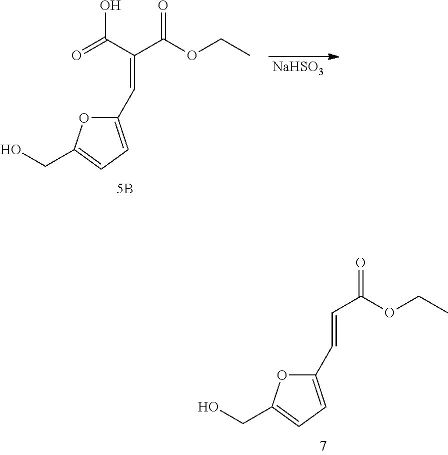

The synthesis of similar furan compounds has been described in relation to the synthesis of pharmaceutical compounds, such as in U.S. Pat. No. 4,507,290, but the methods are much less efficient, i.e. they result in large quantities of by-products, and rely on the use of chemicals that are highly disadvantageous from the safety and/or environmental perspective.

The present invention thus relates to processes such as the ones defined and exemplified above. It also entails various embodiments wherein these processes are combined, e.g. as integrated pathways to convert furfural compounds into useful furan compounds/commodities. In an embodiment of the invention, a high purity Knoevenagel product is produced in ethyl acetate, as described in the foregoing, which is collected using a simple liquid-solid-separation technique, such as filtration, and used for further reactions into the furan commodities, by hydrogenation and subsequent decarboxylation and, optionally, esterification, without any intermediate isolation and/or purification steps.

The present invention also relates to the new products and intermediates that are obtained and/or obtainable by these processes and/or pathways.

These and other aspects of the invention, will become apparent to those skilled in the art, based on the following description and the appending examples.

### DETAILED DESCRIPTION OF THE INVENTION

Hence, a first aspect of the invention concerns a process for producing a furan compound, comprising the steps of:

a) providing a furfural compound selected from the group consisting of hydroxymethylfurfural (HMF), hydroxymethyfurfural esters and hydroxymethylfurfural ethers;
b) reacting the furfural compound with an activated methylene compound comprising at least one electron withdrawing group in the presence of a catalyst to form a Knoevenagel product comprising a substituted furan compound comprising one or more electron-withdrawing groups and one or more carbon-carbon double bonds; and
c) subjecting the Knoevenagel product obtained in step b) to a reaction to form the furan building block, the obtained furan building block comprising at least one intact carboxylic acid functionality selected from the group consisting of —COOH, —C(═O)—O—CH₃ and —C(═O)—O—CH₂CH₃, the reaction being one of the following reactions
c1) a hydrogenation reaction;
c2) a decarboxylation reaction; or
c3) a de-esterification reaction.

As used herein, the term “furfural compound”, encompasses compositions comprising compounds containing the basic furfural moiety, more in particular compounds containing the basic HMF moiety. The IUPAC term for HMF is 5-(hydroxymethyl)-2-furaldehyde. The terms “5-hydroxymethylfurfural”, “hydroxymethylfurfural”, “5-(hydroxymethyl)-2-furaldehyde” and “HMF” are considered synonymous and may be used interchangeably in the context of the present invention. For the purposes of the present invention, the furfural compound may also be an ester or ether of HMF.

As will be apparent to those of average skilled in the art based on the present disclosure, the invention, in certain embodiments, involves subjecting a furfural compound to a so-called Knoevenagel reaction, to yield a product referred to herein as the Knoevenagel product. The product is often an alpha, beta conjugated enone. In one embodiment of the invention, a process as defined herein is provided, wherein the Knoevenagel product has the structure of formula (IV):

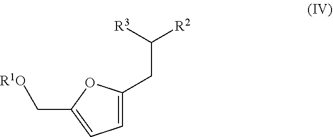

wherein R¹ represents hydrogen, —CH₃, —CH₂CH₃, or —C(═O)CH₃; and R² and R³ represent —C≡N, —COOH, —C(═O)—O—CH₃ or —C(═O)—O—CH₂CH₃.

As will be apparent to those of average skilled in the art based on the present disclosure, the invention, in certain embodiments, involves subjecting the Knoevenagel product to a reaction to yield a product referred to as the ‘furan compound’, whereby the furan compound comprises at least one intact carboxylic acid functionality selected from the group consisting of —COOH, —C(═O)—O—CH₃ and —C(═O)—O—CH₂CH₃. These furan compounds are of particular interest as a chemical intermediate.

In one embodiment of the invention, a process as defined herein comprising a hydrogenation reaction is provided, wherein the furan compound has the structure of formula (Ia):

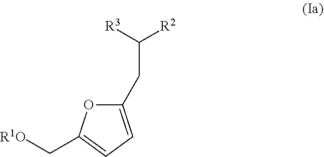

wherein R¹ represents hydrogen, —CH₃, —CH₂CH₃, or —C(═O)CH₃; and R² and R³ represent —C≡N, —COOH, —C(═O)—O—CH₃ or —C(═O)—O—CH₂CH₃.

In another embodiment of the invention, a process as defined herein comprising a decarboxylation or de-esterification reaction, in particular a mono de-esterification reaction is provided, wherein the furan compound has the structure of formula (Ib):

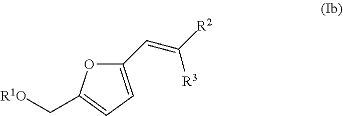

wherein R¹ represents hydrogen, —CH₃, —CH₂CH₃, or —C(═O)CH₃; and R² represents —C≡N, —COOH, —C(═O)—O—CH₃ or —C(═O)—O—CH₂CH₃ and if R³ is present R³ represents C≡N, —COOH, —C(═O)—O—CH₃ or —C(═O)—O—CH₂CH₃.

An example of the compound according to formula (Ia) is:

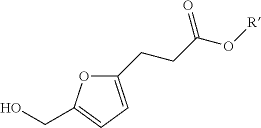

wherein R′ represents —H, —CH₃ or —CH₂—CH₃. Examples of the compound according to formula (Ib) are

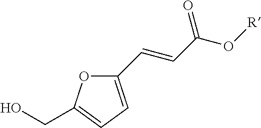

wherein R′ represents —H, —CH₃ or —CH₂—CH₃; and

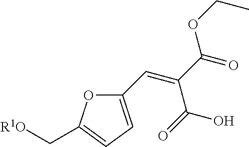

As will be apparent to those of average skilled in the art based on the present disclosure, the invention, in certain embodiments, involves subjecting the furan compound of formula (Ia) or the furan compound of formula (Ib) to further conversions to produce a decarboxylated and esterified furan compound. This furan compound is of particular interest as a chemical intermediate. In one embodiment of the invention, a process as defined herein is provided, wherein a furan compound having the structure of formula (II) is produced:

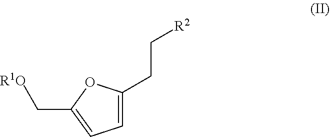

wherein R¹ represents hydrogen, —CH₃, —CH₂CH₃, or —C(═O)CH₃; and R² represents —C≡N, —COOH, —C(═O)—O—CH₃ or —C(═O)—O—CH₂CH₃.

As will be clear from the foregoing based on the present disclosure, an important product/reactant in many processes of the invention is the furfural compound. In one embodiment of the invention, a process as defined herein is provided, wherein the furfural compound has the structure of formula (III):

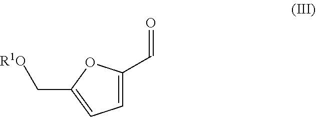

wherein R¹ represents hydrogen, —CH₃, —CH₂CH₃, or —C(═O)CH₃.

For the purposes of the present invention, the furfural compound may in principle be obtained in any way, although furfural compounds obtained from a renewable source is preferred in many embodiments. Renewable sources that may be converted to furfural, include virtually any source of polysaccharides comprising at least one hexose. The extraction of hexoses from such sources and the conversion into the furfural compound may be accomplished in manners known per se by those of average skill in the art, e.g. by the well-known acid catalyzed dehydration of fructose. Hence, for the purposes of the present invention, step a) is not particularly critical; the furfural compound can be produced using any manner known per se and can e.g. be obtained from a (commercial) supplier.

In one preferred embodiment of the invention, the furfural compound provided in step a) is a highly purified furfural compound, typically a furfural compound of more than 90% purity, more preferably of more than 92% purity, more than 94% purity; more than 95% purity; more than 96% purity; more than 97% purity; more than 98% purity; more than 99% purity; or more than 99.5% purity. Such products are typically provided in solid (crystalline) form. Products like these can be sourced directly from commercial suppliers, such as from AVA Biochem. In addition, it is within the purview of those skilled in the art to produce them, starting e.g. from a renewable source of hexose sugars.

In another preferred embodiment of the invention, the furfural compound provided in step a) is a crude or partially purified furfural containing reaction product, e.g. as obtained by the acid catalyzed dehydration of fructose. Such products are typically in the form an aqueous solution or suspension. In an embodiment of the invention, suitable furfural containing aqueous solutions are characterized by the presence of by-products, e.g. at levels of more than 5 wt. %, based on the total dry solids weight, more preferably at levels of more than 6 wt. %; more than 7 wt. %; more than 8 wt. %; more than 9 wt. %; more than 10 wt. %. Said by-products, in an embodiment, typically include any one or any combination of the following: levulinic acid, formic acid, furfural and 5-methylfurfural. Products like these can be sourced directly from commercial suppliers, such as from AVA Biochem. In addition, it is within the purview of those skilled in the art to produce them, starting e.g. from a renewable source of hexose sugars.

Step b) of the process of the invention entails a reaction known in the art as a Knoevenagel reaction or Knoevenagel condensation, which is a nucleophilic addition of an active hydrogen compound to a carbonyl group followed by a dehydration reaction in which a molecule of water is eliminated.

Step b) typically entails the reaction of the furfural compound with an activated methylene compound comprising at least one electron withdrawing group under conditions suitable to produce the Knoevenagel product.

The activated methylene compound employed, may be any methylene compound capable of donating a proton when used with, for example, a basic catalyst. Useful activated methylene compounds may comprise a methylene group (CH₂) associated with one or two electron withdrawing groups such as carboxylic acids, ester or nitrile groups. Suitable examples of such methylene compounds include substituted or unsubstituted malonic acid, its esters or derivatives thereof, as well as, malononitrile or a suitable derivative thereof. Suitable malonic acid esters may include any ester of malonic acid, preferably methyl and ethyl esters of malonic acid.

In one preferred embodiment of the invention the methylene compound is malonic acid. The use of malonic acid is particularly advantageous in that the resulting Knoevenagel product is insoluble in certain organic solvents, such as ethyl acetate. This allows for the process to be set up in such a manner that a high purity Knoevenagel product will directly precipitate from the reaction mixture, which can be collected very easily.

In another preferred embodiment of the invention the methylene compound is a malonic acid ester selected from the group consisting of dimethyl malonate and diethyl malonate. These malonic acid esters can be sourced against significantly lower costs than malonic acid.

In accordance with the invention, in step b), the furfural compound and the activated methylene compound are typically reacted in a molar (furfural compound: methylene compound) ratio within the range of 8/1-1/8, preferably in a ratio within the range of 2/1-1/6, more preferably a ratio within the range of 1/1-1/4, e.g. a ratio of about 1/2.

In accordance with a preferred embodiment of the invention, a suitable solvent is used during step b). The specific solvent to be employed is usually not particularly critical so long as it is capable of significantly dissolving either the furfural compound and the activated methylene compound. Solvents suitable for the purposes of the present invention typically include tetrahydrofuran (THF), ethyl acetate, diethyl ether, toluene, hexanes, water, isopropanol, and mixtures thereof.

As will be understood by those skilled in the art, in case the furfural compound is provided in solid form, step b) will comprise dissolving the furfural compound in the solvent selected for the Knoevenagel reaction. In case the furfural compound is provided in the form of an aqueous solution, a suitable organic solvent may be added to the aqueous solution to yield a combination of water and organic solvent as the reaction medium.

In one particularly preferred embodiment of the invention, ethyl acetate is used as a solvent during step b). This is particularly advantageous since Knoevenagel products in accordance with the invention have been found to precipitate from ethyl acetate in extremely high purity, as already mentioned in the foregoing.

In accordance with a preferred embodiment of the invention, a suitable catalyst is used during step b). The catalyst for the reaction between the furfural compound and the activated methylene compound varies depending upon, for example, the ingredients and reaction conditions. Typically, the catalyst comprises a base capable of extracting a proton from the activated methylene to form the desired substituted or unsubstituted Knoevenagel products or mixture thereof. Bases with a strength (pK_{B}) of less than 6, preferably from about 3 to about 4 are often useful in the present invention. The catalyst can be homogeneous or heterogeneous. Examples of agents that can suitably be used as the catalyst in this process include ethanolamine; 1,2-diamines; dimethylaminopyridine; amino acids such as glycine or alanine; ammonium bicarbonate; MgO; basic alumina; hydrotalcites; oxynitrides; alkali exchanged zeolites; and mixtures thereof. Typically, a suitable heterogeneous catalyst has a high surface area so that a high concentration of basic sites are exposed. Hence, particularly preferred catalysts are selected from the group consisting of supported organic bases, e.g. 1,2-diamines supported on poly(styrene), 3-aminopropyl-functionalized silica, dimethylaminopyridine on poly(styrene); a solid base, such as MgO, basic alumina, hydrotalcites, oxynitrides, alkali exchanged zeolites; and mixtures thereof. In a preferred embodiment of the invention the catalyst used in step b) is selected from the group consisting of ethanolamine, 1,2-diamines, such as ethylenediamine, propane 1,2-diamine or cyclohexane 1,2-diamine (cis or trans), on a solid (poly(styrene)) support; 3-aminopropyl-functionalized silica or other base-functionalized oxide materials, dimethylaminopyridine on poly(styrene), MgO, hydrotalcites, oxynitrides, alkali exchanged zeolites, and mixtures thereof. In one preferred embodiment, the catalyst is selected from the group of ethanolamine, ethylenediamine, propane 1,2-diamine or cyclohexane 1,2-diamine (cis or trans), supported on poly(styrene), most preferably ethylenediamine on poly(styrene).

Ammonium bicarbonate has the advantage that it is environmentally friendly (meeting the international green chemistry standards) and is therefore particularly preferred. The present inventors have established that the use of ammonium bicarbonate results in a reaction mechanism involving the formation of the following intermediate that is reactive towards activated methylene compounds, such as, in particular, malonic acid and malonic acid esters:

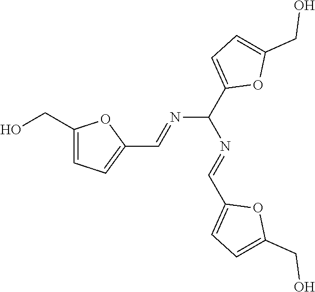 [SMILES: OCc1ccc(/C=N/C(/N=C/c2ccc(CO)o2)c2ccc(CO)o2)o1]

The reaction between the furfural compound and activated methylene compound may occur in any convenient vessel, at any convenient temperature, and any convenient pressure. The reaction time, temperature, and pressure to be employed will vary depending upon the ingredients, desired products, and other reaction conditions. As will be understood, the higher the temperature the faster the reaction will occur. In accordance with the invention, step b) is typically carried out by contacting the furfural compound and the activated methylene compound, optionally in the presence of the catalyst, in a suitable reactor at a temperature within the range of 40-120° C., preferably within the range of 50-100° C., most preferably within the range of 60-90° C. In accordance with the invention, during step b), the system is at a pressure within the range of 1-5 Bar, more preferably within the range of 1-3 Bar, most preferably within the range of 1-2 Bar. In accordance with the invention, step b) can be carried out in closed pressure reactors or vessels equipped with condensers. Pressures typically range from atmospheric to elevated pressures generated autogenously in closed vessels.

Hence, in an embodiment of the invention, a process as defined herein before is provided, wherein step b) comprises the steps of:

b1) combining the furfural compound, the activated methylene compound and the catalyst in a suitable solvent, to produce a liquid reaction mixture;
b2) keeping the liquid reaction mixture under conditions under which the Knoevenagel reaction proceeds.

The reaction is carried out for a period of time sufficient to effect conversion under the chosen conditions. The reaction time can range from several minutes to a number of hours, preferably from 30 minutes to 5 hours, most preferably from 1-4 hours. Conversion of the reactants into the Knoevenagel product is preferably up to about 100% and most preferably from about 70% to about 100%. Selectivity for the target Knoevenagel product, is preferably from about 20% to 100% and most preferably from about 70% to 100%.

In certain embodiments of the invention, step b), may comprise separation and/or isolation of the Knoevenagel product from the reaction mixture. In a preferred embodiment of the invention, step b2) is followed by the separation, isolation or purification of the Knoevenagel product from the reaction mixture. Suitable techniques to accomplish this are within the common general knowledge of the person skilled in the art.

In one embodiment of the invention, the activated methylene compound is malonic acid and the solvent is ethyl acetate, and step b2) comprises the additional step of separating precipitated Knoevenagel product from the reaction mixture by a solid-liquid separation technique. In one embodiment of the invention, a process as defined herein is provided, wherein step b2) comprises keeping the liquid reaction mixture under conditions under which the Knoevenagel reaction proceeds, followed by subjecting the reaction mixture to a filtration step, so as to collect the precipitated Knoevenagel product. In an embodiment of the invention, the Knoevenagel product accordingly obtained is used as the starting material for step c) without any further purification being performed.

In one embodiment of the invention, the activated methylene compound is malonic acid ester and the solvent is ethyl acetate, and step b2) comprises the additional steps of subjecting the Knoevenagel product to de-esterification treatment followed by separating the precipitating/precipitated Knoevenagel product from the reaction mixture by a solid-liquid separation technique. The de-esterification may suitably be accomplished by addition of a catalytic amount of aqueous acid solution to the reaction mixture. A preferred acid for this purpose is sulfuric acid, which can typically be used in an amount of e.g. 0.5-10 mol. %, preferably 1-5 mol. %, of the Knoevenagel (di-ester). Hence, in one embodiment of the invention, a process as defined herein is provided, wherein step b2) comprises keeping the liquid reaction mixture under conditions under which the Knoevenagel reaction proceeds, followed by the addition of a catalytic amount of an aqueous acid solution, preferably aqueous sulfuric acid solution to the reaction mixture and subsequently subjecting the reaction mixture to a filtration step, so as to collect the precipitated Knoevenagel product. In an embodiment of the invention, the Knoevenagel product accordingly obtained is used as the starting material for step c) without any further purification being performed.

In accordance with the present invention, the Knoevenagel product obtained in step b) is subjected to a hydrogenation reaction (referred to herein as step cl)), to a decarboxylation reaction (referred to herein as step c2)) or to a de-esterification reaction, for example a mono de-esterification reaction (referred to herein as step c 3)).

In case the Knoevenagel product obtained in step b) is subjected to a hydrogenation reaction, the hydrogenation is typically carried out by contacting the Knoevenagel product with hydrogen gas in the presence of a hydrogenation catalyst. Hence, processes are provided in accordance with the invention wherein step c1) comprises:

c1a) combining the Knoevenagel product and a catalyst in a suitable solvent, to produce a liquid reaction mixture;
c1b) contacting the liquid reaction mixture with hydrogen under conditions under which the hydrogenation proceeds, preferably at a temperature within the range of 20-120° C. and a pressure within the range of 1-10 bar.

A suitable hydrogenation catalyst may in particular be selected from the group of nickel catalysts, such as Raney nickel, or nickel nanoparticles, either in solution or on a carrier material, palladium, (e.g. on active coal or on another carrier material or in the form of nanoparticles), ruthenium (on carbon, in the form of nanoparticles or on another carrier material), rhodium (on carbon, in the form of nanoparticles or on another carrier material), platinum (on carbon, in the form of nanoparticles or on another carrier material), iron (on carbon, in the form of nanoparticles or on another carrier material), gold (on carbon, in the form of nanoparticles or on other carrier material) or copper chromite. Nickel catalysts are preferred. Especially preferred is the use of Raney nickel or the use of nickel nanoparticles. It is also possible to use mixtures of catalysts. As used herein, the term ‘nanoparticles’ means particles of a solid or semi-solid material having a weight average diameter, as determinable by scanning electron microscopy (SEM) or transmission electron microscopy (TEM) in the range of 1-1000 nm, in particular in the range of 5-500 nm. In certain preferred embodiments of the invention, step c) comprises hydrogenation of the Knoevenagel product in the presence of Raney nickel.

In certain embodiments of the invention, the hydrogenation of the Knoevenagel product is typically carried out in a protic solvent (e.g. an inert alcohol, such as methanol, ethanol or 1-propanol, a cycloalkane, such as cyclohexane, or in dimethoxymethane) or in water or an aqueous solvent. The use of water is particularly preferred.

In certain embodiments of the invention, the molar ratio hydrogen gas to Knoevenagel product is typically at least stoichiometric. Preferably an excess hydrogen gas is used. In particular, the molar ratio may be in the range of 10 to 2000.

The Knoevenagel product to catalyst ratio (w/w) is usually chosen between 1:1 and 500:1; a preferred range is from 4:1 to 50:1.

The hydrogenation may conveniently be carried out in a continuous stirred tank (CSTR) or a flow reactor. The reaction is typically carried out at a temperature and pressure and for a contact time sufficient to effect conversion. Reaction temperature can typically range from 30° C. to 600° C. In certain preferred embodiments of the invention, step c) comprises hydrogenation of the Knoevenagel product in the presence of Raney nickel at a temperature within the range of 20° C. to 120° C., preferably within the range of 30 to 100° C., preferably within the range of 40-90° C., more preferably within the range of 50-85° C., and most preferably within the range of 60-80° C. The hydrogen pressure can typically range from 1 to 120 bar. In certain preferred embodiments of the invention, step c1a) comprises hydrogenation of the Knoevenagel product in the presence of Raney nickel at a hydrogen pressure within the range of 1-10 Bar, preferably within the range of 1.25-7.5 Bar, more preferably within the range of 1.5-5 Bar, more preferably within the range of 1.75-4 Bar, most preferably within the range of 2-3 Bar. The reaction is carried for a period of time sufficient to effect conversion under the chosen conditions. The reaction time can range from several minutes to a number of hours, preferably from 30 minutes to 10 hours, most preferably from 1-5 hours. Conversion of the Knoevenagel product is preferably from about 20% to about 100% and most preferably from about 70% to about 100%. Selectivity for the target furan compound, is preferably from about 20% to 100% and most preferably from about 70% to 100%.

In case the Knoevenagel product obtained in step b) is subjected to a decarboxylation step, i.e. according to step c2), the decarboxylation is for example carried out in the presence of pyridine.

In a preferred embodiment of the invention, step c2) comprises a reactive destillation. Gradually evaporation of the Knoevenagel product results in decarboxylation producing a compound with a structure of formula (Ib).

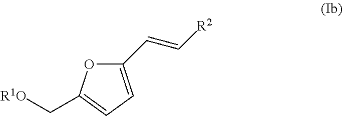

wherein R¹ represents hydrogen, —CH₃, —CH₂CH₃, or —C(═O)CH₃ and R² represents —C≡N, —COOH, —C(═O)—O—CH₃ or —C(═O)—O—CH₂CH₃.
A particularly preferred compound according to formula (Ib) is given below:

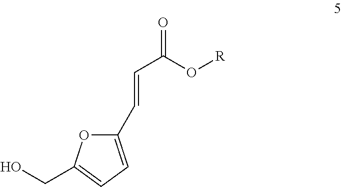

wherein R represents hydrogen, —CH₃ or —CH₂CH₃.

In case the Knoevenagel product obtained in step b) is subjected to a de-esterification step, i.e. according to step c3), the de-esterification is typically carried out by adding one equivalent of KOH followed by the addition of NaHSO₃.

In certain embodiments of the invention, step c), (either step c1), step c2) or step c3)) may comprise separation and/or isolation of the furan compound from the reaction mixture, by any suitable technique known by the person skilled in the art. Embodiments are also envisaged, wherein the reaction mixture produced in step c) (either step c1), step c2) or step c3)) is immediately used for further conversion reactions, such as those described here below as steps d) and e), i.e. without intermediate isolation or purification, other than e.g. separating solids from the liquid. In a particularly preferred embodiment of the invention, step c) (either step c1), step c2) or step c3)) and step d) are performed as a ‘one-pot process’.

As defined in the foregoing, in certain embodiments of the invention, a process as defined herein is provided, further comprising a step d) of decarboxylation of the furan compound obtained in step c), for example in step c1), step c2) or step c3). This step d) typically entails the reaction of the furan compound obtained in step c), for example in step c1), step c2) or step c3), with a homogeneous acid catalyst under conditions suitable to remove a carboxylic acid moiety.

In preferred embodiments of the invention, a process is provided in which step c) comprises a hydrogenation reaction according to step c1) further comprising a step d) of decarboxylation of the furan compound obtained in step c1).

In alternative preferred embodiments of the invention, a process is provided in which step c) comprises a de-esterification reaction according to step c3) further comprising a step d) of decarboxylation of the furan compound obtained in step c3).

It is envisaged that virtually any strong acid can suitably be used for the decarboxylation reaction. Particular, non-limiting, examples of suitable strong acids include hydrochloric acid and sulphuric acid. The use of sulphuric acid may be preferred in certain embodiments of the invention, as the resulting sulphate salt will be relatively easy to separate from product.

In typical embodiments of the invention step d) is typically performed in an aqueous solution of the homogeneous acid catalyst, having a pH of below 1.

Hence, in certain preferred embodiments of the invention, a process is provided as defined herein before, wherein step d) comprises combining a quantity of the furan compound produced in step c), in an aqueous solution, with the homogenous acid catalyst, to produce a liquid reaction mixture, while keeping the liquid reaction mixture under conditions under which the acid-catalyzed decarboxylation reaction proceeds. In certain preferred embodiments of the invention, a process is provided as defined herein before, wherein step d) comprises providing a quantity of the reaction mixture produced in step c) and adding a quantity of the homogenous acid catalyst, to produce a liquid reaction mixture, while keeping the liquid reaction mixture under conditions under which the acid-catalyzed decarboxylation reaction proceeds.

In accordance with the invention, step d) is typically carried out in a suitable reactor at a temperature within the range of 10-90° C., preferably within the range of 15-85° C., most preferably within the range of 20-80° C. In accordance with the invention, during step d), the system is typically at ambient pressure. The reaction is typically carried out by the addition of the acid to the reaction mixture and keeping the mixture under suitable conditions for a period of time sufficient to effect conversion. The reaction time can range from 10 minutes to 100 hours, preferably from 30 minutes to 72 hours. The precise reaction rate is influenced by the rate of addition of the acid to the reaction mixture, which generates heat. The reaction rate can also be increased by external heating.

Conversion of the furan compound into the target decarboxylated furan is preferably up to about 100% and most preferably from about 70% to about 100%. Selectivity for the target product, is preferably from about 20% to 100% and most preferably from about 70% to 100%.

In certain embodiments of the invention, step d), may comprise the separation, isolation or purification of the decarboxylated furan compound from the reaction mixture by any suitable technique known by the person skilled in the art. Embodiments are however also envisaged, wherein the reaction mixture produced in step d) is directly used for further conversion, e.g. to an esterification step e) as described here after, without intermediate isolation or purification, other than e.g. separating solids from the liquid. In a particularly preferred embodiment of the invention, step d) and step e) are performed as a ‘one-pot process’.

In certain embodiments of the invention, a process as defined herein is provided, further comprising a step e) of esterifying the decarboxylated reaction product of step d). This step e) typically entails the reaction of the decarboxylated reaction product of step d) with an alcohol under conditions under which an esterification reaction occurs.

In accordance with the invention, the alcohol is typically selected from the group consisting of ethanol, methanol, n-propanol, i-propanol, n-butanol and t-butanol. Most preferably the alcohol is methanol or ethanol.

In accordance with the invention, in step e), the decarboxylated reaction product of step d) and the alcohol are typically reacted in a ratio within the range of 1/100-1/1 preferably a ratio within the range of 1/80-1/25, most preferably a ration within the range of 1/70-1/50.

In accordance with a preferred embodiment of the invention, a suitable catalyst is used during step e). Catalysts suitable for use in step e) typically include homogeneous acid catalysts. It is envisaged that virtually any strong acid can suitably be used for the esterification reaction. Particular, non-limiting, examples of suitable strong acids include hydrochloric acid and sulphuric acid. The use of sulphuric acid may be preferred in certain embodiments of the invention, as the resulting sulphate salt will be relatively easy to separate from product. In accordance with the invention, in step e), the catalyst is typically used in an amount of 0.1-10% (w/w) of the amount of the decarboxylated reaction product of step d), preferably in an amount of 0.25-5% (w/w), most preferably in an amount of 0.5-2% (w/w).

In accordance with the invention, step e), is typically carried out by contacting the decarboxylated reaction product and the alcohol, optionally in the presence of the catalyst, in a suitable reactor under reflux.

The reaction is carried for a period of time sufficient to effect conversion under the chosen conditions. In many embodiments, the reaction time can typically range from 1 minute to 10 hours, preferably from 10 minutes to 5.hours, most preferably from 30 minutes to 3 hours.

Conversion of the reactants into the target furan ester can be up to about 100% and most preferably ranges from about 70% to about 100%. Selectivity for the target furan ester, is preferably from about 20% to 100% and most preferably from about 70% to 100%.

In certain embodiments of the invention, step e), may comprise separation and/or isolation of certain components present in the reaction mixture. In a preferred embodiment of the invention, step e), may comprise the separation, isolation or purification of the furan ester from the reaction mixture by any suitable technique known by the person skilled in the art. In one embodiment of the invention, step e), comprises the step of treating the reaction mixture by extraction with an organic solvent, such as MBTE, followed by evaporation of the organic solvent, yielding the furan ester.

As will be evident from the foregoing, the present invention in part resides in the realization that the various reactions described here above can be integrated to produce new and unique chemical processes, that have particular utility in conversions of biomass into versatile furan commodities. Various aspects of the present invention concern such specific processes.

In one such aspect, a process is provided for producing a furan compound according to formula (I) as defined herein, comprising the steps of:

a) providing a furfural compound selected from the group consisting of hydroxymethylfurfural (HMF), hydroxymethyfurfural esters and hydroxymethylfurfural ethers;
b) reacting the furfural compound with an activated methylene compound comprising at least one electron withdrawing group in the presence of a catalyst to form a Knoevenagel product comprising a substituted furan compound comprising one or more electron-withdrawing groups and one or more carbon-carbon double bonds; and
c) subjecting the Knoevenagel product obtained in step b) to a hydrogenation reaction, a decarboxylation reaction or a de-esterification, for example a mono de-esterification reaction to form the furan compound.

In one aspect, a process is provided for producing a furan compound according to formula (I) as defined herein, comprising the steps of:

a) providing a furfural compound selected from the group consisting of hydroxymethylfurfural (HMF), hydroxymethyfurfural esters and hydroxymethylfurfural ethers, in solid, e.g. in crystalline form;
b) reacting the furfural compound, in ethyl acetate, with malonic acid in the presence of a catalyst to form a Knoevenagel product comprising a substituted furan compound comprising one or more electron-withdrawing groups and one or more carbon-carbon double bonds, whereby the Knoevenagel product precipitates from the ethyl acetate, following which the Knoevenagel product is separated from the liquid; and
c) placing the Knoevenagel product obtained in step b) in an aqueous solvent and subjecting it to a hydrogenation reaction, to a decarboxylation reaction or to a de-esterification reaction, for example a mono de-esterification reaction to form the furan compound.

In a preferred process step c) comprises the step of placing the Knoevenagel product obtained in step b) in an aqueous solvent and subjecting it to a hydrogenation reaction to form the furan compound.

In an embodiment, the above-defined process is followed by the subsequent steps of d) reacting the furan compound obtained in step c) with a homogeneous acid catalyst under conditions suitable to remove a carboxylic acid moiety; and e) reacting the decarboxylated reaction product of step d) with an alcohol under conditions under which an esterification reaction occurs. In a preferred embodiment said steps c), d) and e) are performed without isolation of the intermediates, preferably wherein steps c), d) and e) are performed as a one-pot-process.

In one aspect, a process is provided for producing a furan compound according to formula (I) as defined herein, comprising the steps of:

a) providing a furfural compound selected from the group consisting of hydroxymethylfurfural (HMF), hydroxymethyfurfural esters and hydroxymethylfurfural ethers, in solid, e.g. in crystalline form;
b) reacting the furfural compound, in ethyl acetate, with a malonic acid ester in the presence of a catalyst to form a Knoevenagel product comprising a substituted furan compound comprising one or more electron-withdrawing groups and one or more carbon-carbon double bonds, followed by acid catalysed hydrolysis of the ester, whereby the Knoevenagel product precipitates from the ethyl acetate, following which the Knoevenagel product is separated from the liquid; and
c) placing the Knoevenagel product obtained in step b) in an aqueous solvent and subjecting it to a hydrogenation reaction, a decarboxylation reaction or a de-esterification reaction, for example a mono de-esterification reaction to form the furan compound. Preferably, the Knoevenagel product obtained in step b) is placed in an aqueous solvent and subjected to a hydrogenation reaction to form the furan compound.

In an embodiment, the above-defined process is followed by the subsequent steps of d) reacting the furan compound obtained in step c) with a homogeneous acid catalyst under conditions suitable to remove a carboxylic acid moiety; and e) reacting the decarboxylated reaction product of step d) with an alcohol under conditions under which an esterification reaction occurs. In a preferred embodiment said steps c), d) and e) are performed without isolation of the intermediates, preferably wherein steps c), d) and e) are performed as a one-pot-process.

In one aspect, a process is provided for producing a furan compound according to formula (I) as defined herein, comprising the steps of:

a) providing a furfural compound selected from the group consisting of hydroxymethylfurfural (HMF), hydroxymethyfurfural esters and hydroxymethylfurfural ethers, in the form of an aqueous suspension, e.g. the crude or partially purified reaction mixture obtained by the acid-catalyzed dehydration of a hexose sugar;
b) reacting the furfural compound, in a medium comprising an organic solvent and water, with a malonic acid or an ester thereof in the presence of a catalyst to form a Knoevenagel product comprising a substituted furan compound comprising one or more electron-withdrawing groups and one or more carbon-carbon double bonds; followed by isolation or purification of the Knoevenagel product; and
c) placing the Knoevenagel product obtained in step b) in an aqueous solvent and subjecting it to a hydrogenation reaction, a decarboxylation reaction or a de-esterification reaction, for example a mono de-esterification reaction to form the furan compound. Preferably, the Knoevenagel product obtained in step b) is placed in an aqueous solvent and subjected to a hydrogenation reaction to form the furan compound

In an embodiment, the above-defined process is followed by the subsequent steps of d) reacting the furan compound obtained in step c) with a homogeneous acid catalyst under conditions suitable to remove a carboxylic acid moiety; and e) reacting the decarboxylated reaction product of step d) with an alcohol under conditions under which an esterification reaction occurs. In a preferred embodiment said steps c), d) and e) are performed without isolation of the intermediates, preferably wherein steps c), d) and e) are performed as a one-pot-process.

Further aspects of the invention relate to the reaction products obtainable by any one of the steps, processes and/or pathways as defined in any of the foregoing.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

Furthermore, for a proper understanding of this document and in its claims, it is to be understood that the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXAMPLES

### Example 1: Conversion of HMF (solid) into Knoevenagel Product Using Malonic Acid

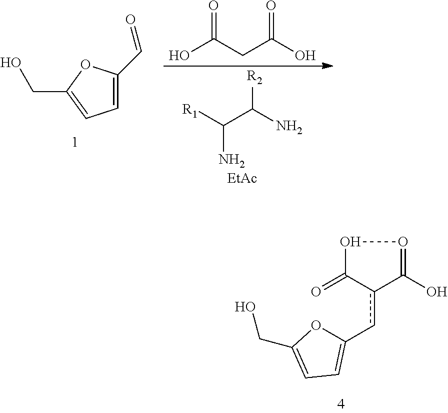

Add HMF (1, 8.78 g, 69.6 mmol), malonic acid (13.99 g, 134.4 mmol), diluted ethylenediamine (2 drops in 150 ml ethyl acetate) in a round-bottom flask and stir the mixture until it form a homogeneous solution.

Submerge the flask with the prepared solution in a water bath at 80° Celsius and react for 180 minutes while stirring and observe the formation of a yellow brown solid.

Use a vacuum filtration setup to separate the solid from the reaction mixture and wash the solid with cold ethyl acetate. Collect the product (4, 12,8g, yield 87%; purity 98%).

If further purification is desired because lots of imine formation took place, the product can be dissolved in THF, followed by a decantation step.

After evaporation of the solvent the purified product can be isolated. The used THF can be recycled for further use.

| | HMF (1) | Knoevenagel product (4) |
|---|---|---|
| IUPAC | 5-(hydroxymethyl)furan-2- | 2-((5-(hydroxymethyl)furan-2- |
| | carbaldehyde | yl)methylene)malonic acid |
| CAS nr | 67-47-0 | — |
| MW (g/mol) | 126.11 | 212.16 |
| mp (° C.) | 32-35 | 166 (degradation) |
| Appearance | brown solid | Yellow/beige solid |

### Example 2: Conversion of HMF (Aqueous Solution) into Knoevenagel Product Using Malonic Acid

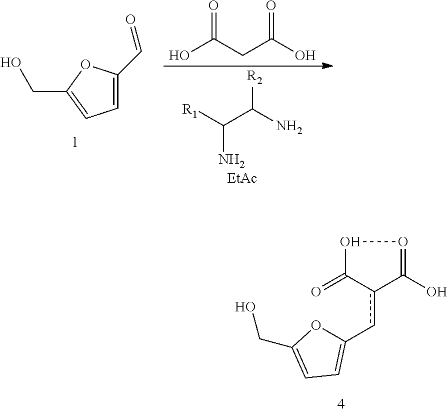

Add aqueous HMF (1, 20 ml, 20 mmol) and diluted ethylenediamine (4 drops in 40 ml ethyl acetate) in a round-bottom flask and stir the mixture until it forms a homogeneous solution.

Also prepare a solution of malonic acid (2.1 g, 20 mmol) in 20 ml demineralized water. Transfer the malonic solution to a dropping funnel and connect this to the flask.

Submerge the flask with the prepared solution in a water bath at 60° Celsius, make sure the dropping funnel drops slowly, and react for 24 hours while stirring vigorously.

After 24 hours add diluted ethylenediamine (2 drops in 20 ml ethyl acetate) and a solution of malonic acid (2.1 g, 20 mmol) in 20 ml demineralized water drop wise to the reaction mixture and react for another 24 hours.

When the reaction time has been reached transfer the reaction mixture to a separatory funnel and add concentrated sulfuric acid to bring the mixture below a pH of 1. Extract three times with 50 ml ethyl acetate. Dry the organic layer over magnesium sulfate, followed by vacuum filtration of the salt. Wash the salt with dry ethyl acetate.

Continue with the evaporation of the solvent and gather the product (4, 2.3 g, yield 53%; purity 75%).

For transfer from the flask to a container acetone can be used and be evaporated after.

| | HMF (1) | Knoevenagel product (4) |
|---|---|---|
| IUPAC | 5-(hydroxymethyl)furan-2- | 2-((5-(hydroxymethyl)furan-2- |
| | carbaldehyde | yl)methylene)malonic acid |
| CAS nr | 67-47-0 | — |
| MW (g/mol) | 126.11 | 212.16 |
| mp (° C.) | 32-35 | 166 (degradation) |
| Appearance | brown solution | Yellow/beige solid |

### Example 3: Conversion of HMF (Solid) into Knoevenagel Product Using Diethyl Malonate

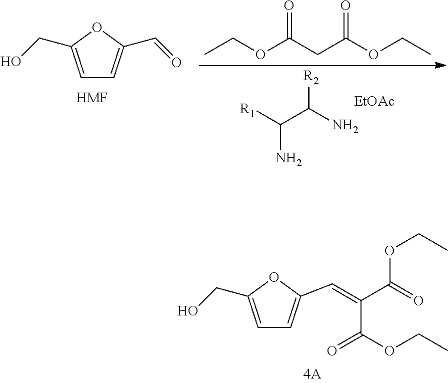

Add HMF (1, 2.52 g, 20 mmol), diethylmalonate (6.4 g, 6.1 ml, 40 mmol), diluted ethylenediamine (2 drops in 40 ml ethyl acetate) in a round-bottom flask and stir the mixture until it forms a homogeneous solution.

Submerge the flask with the prepared solution in a water bath at 80° Celsius and react for 24 hours while stirring.

When the reaction time has been reached, dissolve the reaction mixture in 200 ml ethyl acetate and transfer to a separatory funnel.

Prepare 100 ml demineralized water with two drops of concentrated sulfuric acid and wash the organic layer three times with the aqueous solution.

Separate the organic layer from the aqueous one and dry the ethyl acetate over magnesium sulfate, followed by evaporation of the solvent.

The product was collected after silica purification using Petroleum ether 60-80/Ethyl acetate as a mobile phase. (4a, 4.1 g, yield 77%; purity 99%).

| | HMF(1) | Knoevenagel product (4A) |
|---|---|---|
| IUPAC | 5-(hydroxymethyl)furan-2- | 2-((5-(hydroxymethyl)furan-2- |
| | carbaldehyde | yl)methylene)malonic acid di- |
| | | ethyl ester |
| CAS nr | 67-47-0 | — |
| MW (g/mol) | 126.11 | 268.09 |
| mp (° C.) | 32-35 | — |
| Appearance | brown solid | Brown oil |

### Example 4: Conversion of HMF (Aqueous Solution) into Knoevenagel Product Using Diethyl Malonate

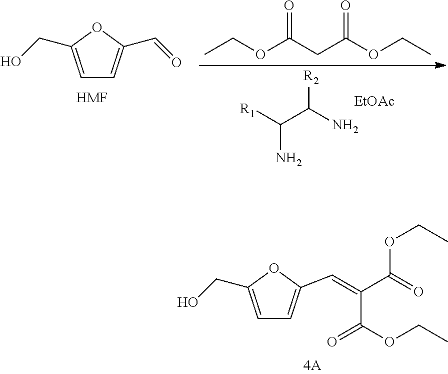

Add aqueous HMF (1, 20 ml, 20 mmol), diethylmalonate (6.4 g, 6.1 ml, 40 mmol) and diluted ethylenediamine (4 drops in 20 ml ethyl acetate) in a round-bottom flask and stir the mixture until it forms a homogeneous solution.

Submerge the flask with the prepared solution in a water bath at 80° Celsius and react for 48 hours while stirring.

When the reaction time has been reached, poor the reaction mixture into 200 ml ethyl acetate and transfer to a separation funnel after filtration.

Wash the organic layer three times with the demineralized water.

Separate the organic layer from the aqueous one and dry the ethyl acetate over magnesium sulfate, followed by evaporation of the solvent.

The product was collected after silica purification using Petroleum ether 60-80/Ethyl acetate as a mobile phase. (4A, 4.0 g, yield 75%; purity 98%).

| | HMF (1) | Knoevenagel product (4A) |
|---|---|---|
| IUPAC | 5-(hydroxymethyl)furan-2- | 2-((5-(hydroxymethyl)furan-2- |
| | carbaldehyde | yl)methylene)malonic acid di- |
| | | ethyl ester |
| CAS nr | 67-47-0 | — |
| MW (g/mol) | 126.11 | 268.09 |
| mp (° C.) | 32-35 | — |
| Appearance | brown solution | Brown oil |

### Example 5: De-esterification of Knoevenagel Product

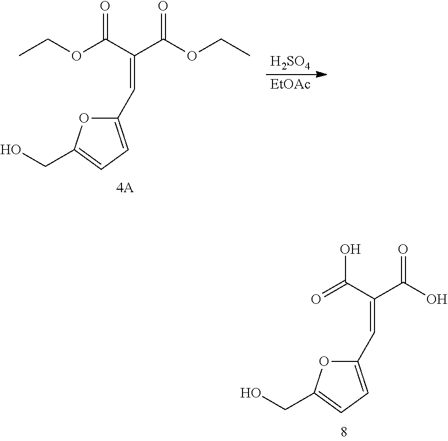

Add HMF-biethylester (0.27 g, 1.0 mmol), as prepared in example 3 or 4, diluted sulfuric acid (0.2 ml, 1M) and ethyl acetate (2 ml) in a round bottom flask (5 ml).

Connect a cooler to the flask and submerge the flask in an oil bath, heated at 110° C. with a magnetic hot plate.

Reflux the mixture 96 hours and observe precipitation.

When the reaction time has been reached, transfer the reaction mixture to a separatory funnel and add 100 ml of ethyl acetate.

Extract the organic layer three times with 50 ml demineralised water and combine the extracts.

Wash the extract two times with 50 ml ethyl acetate and evaporate the water layer with the rotary evaporator.

A brown solid can be collected.

| Knoevenagel product (4A) | De-esterified product (8) | |
|---|---|---|
| IUPAC | 2-((5-(hydroxymethyl)furan-2- | 2-((5-(hydroxymethyl)furan-2- |
| | yl)methylene)malonic acid di- | yl)methylene)malonic acid |
| | ethyl ester | |
| CAS nr | — | 67-47-0 |
| MW (g/mol) | 180.16 | 126.11 |
| mp (° C.) | 203−205 | 32-35 |
| Appearance | White crystals | Brown Solid |

### Example 6: Hydrogenation of the Knoevenagel Product

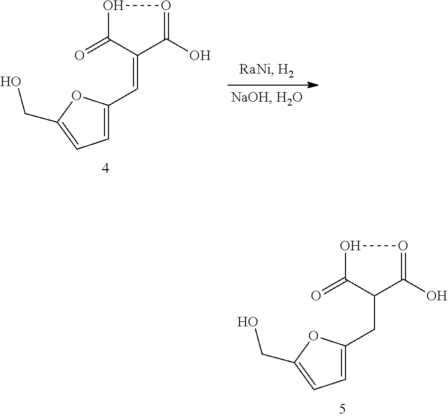

The Knoevenagel product (4) as prepared in example 1 (5.0 gram, 23.5 mmol) is dissolved in aqueous NaOH solution (300 ml, 2 M) and 1 spoon of RaneyNickel (+/−1.2 gram) is added. The mixture is transferred into a Parr shaker type hydrogenation reactor and allowed to react for 3 hours at 80° C. and at a pressure of 3 Bar H₂. Subsequently, the reaction mixture is decanted and the remaining RaNi is washed with demi water. The product (5) has to be processed into the decarboxylated product (3) immediately, following the procedure set out in example 6.

| | Knoevenagel product (4) | Hydrogenated product (5) |
|---|---|---|
| IUPAC | 2-((5-(hydroxymethyl)furan-2- | 2-((5-(hydroxymethyl)furan)- |
| | yl)methylene)malonic acid | ethylene)malonic acid |
| CAS nr | — | — |
| MW (g/mol) | 212.16 | 214.16 |
| mp (° C.) | 166 (degradation) | — |
| Appearance | Yellow/beige solid | — |

### Example 7: Decarboxylation

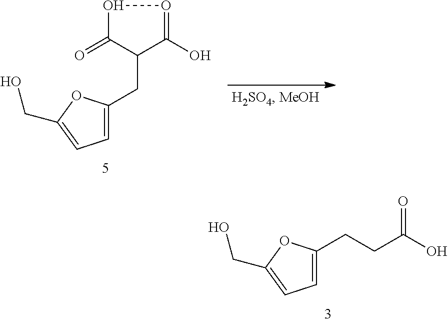

The reaction mixture as prepared in example 6 is heated to 80° C. Under vigorous stirring, concentrated sulphuric acid is added dropwise to lower the pH to 0. A colour change is observed from light/pale orange (pH 6) to red (pH 0). Gas formation is observed during the process. The solution is neutralized with aqueous NaOH-solution (12 M) to pH 6-7. Water is subsequently removed by freeze drying. The product obtained is extracted four times with 50 ml ethanol. The organic layers are combined and evaporated, yielding the product as a yellow oil ((3), 3.0 gram, 17.6 mmol, 75% relative to (4)).

The washing with ethanol results in substantial loss of product. It was established that it feasible to use the crude product of the decarboxylation reaction in the subsequent esterification, following the procedure described in example 7, resulting in a much higher yield (92% of the ester (6) relative to (4)).

| | Hydrogenated product (5) | Decarboxylated product (3) |
|---|---|---|
| IUPAC | 2-((5-(hydroxymethyl)furan) | 3-(5- |
| | ethylene)malonic acid | (hydroxymethyl)furan)propanoic |
| | | acid |
| CAS nr | — | — |
| MW (g/mol) | 214.16 | 170.16 |
| mp (° C.) | — | — |
| Appearance | — | Yellow oil |

### Example 8: Esterification

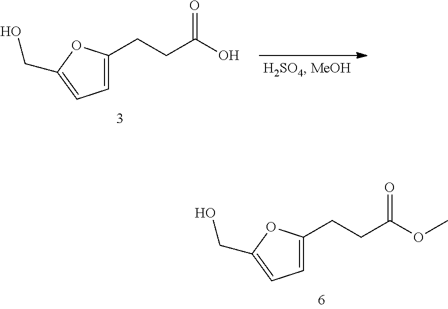

The crude reaction product as prepared in example 7 (containing the decarboxylated product (3)) is dissolved in a minimal volume of hot ethanol (approximately 50 ml), whereafter 3 ml concentrated hydrochloric acid is added. The mixture is heated to reflux temperature and the reaction is monitored using TLC/HPLC. After completion of the reaction, the reaction product is neutralized to a pH within the range of 6-7 with an aqueous NaOH-solution (1M). The mixture is subjected to vacuum filtration and the precipitated salts are washed with cold EtOH (50 ml). The solution is placed in a separation funnel and 250 ml of a saturated sodium bicarbonate solution is added. Extract four times with 100 m MTBE. Combine the organic layers and evaporate the solvent to obtain the product ((6), 8.23 g, 92% relative to (4)).

| | Decarboxylated product (3) | ester (6) |
|---|---|---|
| IUPAC | 3-(5- | 2-((5-(hydroxymethyl)furan)- |
| | (hydroxymethyl)furan)propanoic | ethylene)malonic acid methyl ester |
| | acid | |
| CAS nr | — | — |
| MW (g/mol) | 170.16 | 170.16 |
| mp (° C.) | — | 203-205 |
| Appearance | Yellow oil | Yellow/beige solid |

### Example 9: Conversion of HMF (Solid) into Knoevenagel Product Using Diethyl Malonate

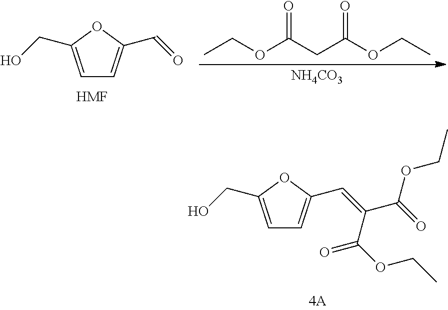

Add HMF (1, 2.52 g, 20 mmol) and diethylmalonate (3.2, 3.0 ml, 20 mmol) in a round-bottom flask and stir the mixture until it forms a homogeneous solution. Heat this mixture under constant stirring to 90° Celsius. As the temperature is reached, add ammonium bicarbonate (0.25 g, 3 mmol) to the mixture.

The solid was kept for 2 hours at 90° Celsius for complete conversion while stirring.

### Example 10: Mono De-esterification of Knoevenagel Product

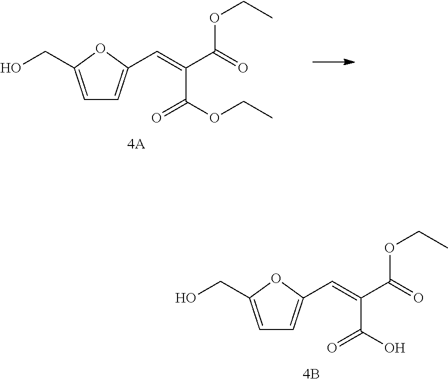 [SMILES: CCOC(=O)C(=Cc1ccc(CO)o1)C(=O)OCC ; CCOC(=O)/C(=C/c1ccc(CO)o1)C(=O)O]

Weigh out HMF-biethylester (4A, 3.43 g, 12 mmol), as prepared in example 9. Dissolve the HMF-biethylester in 20 ml THF and add 100 ml of water in a round-bottom flask and cool mixture to 0° C. Celsius in an ice bath.

Add dropwise 5 ml 2.5 M KOH solution (12.5 mmol). Keep the solution for 5 hours at 0° C. while stirring for complete mono-hydrolysis.

Add 5 ml 2.5 M HCl solution until the mixture is neutralized still at 0° C. Saturate the solution with NaCl. Extract the product obtained four times with 15 ml ethyl acetate. Combine the organic layers and evaporate.

### Example 11: Mono De-carboxylation of De-esterified Knoevenagel Product

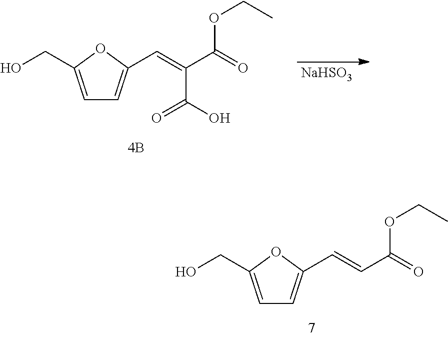

Add HMF-monoethylester (4B, 6.9 g, 26 mmol) and sodium bisulfite (3.0 g, 28 mmol) in a round-bottom flask to 20 ml water and stir. Warm the mixture until no more gas evolves. Add 20 ml of a mixture of 30% hydrogen peroxide solution and 1M sulfuric acid and reflux for one hour. Pour the resulting solution over crushed ice and filter.

### Example 12: Conversion of HMF (Solid) into Knoevenagel Product Using Malonic Acid and Decarboxylation of the Knoevenagel Product

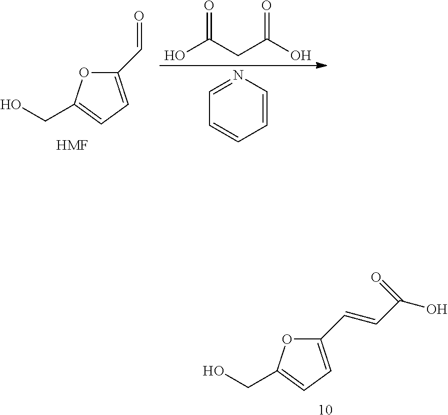

Add crystalline HMF (1.4 g 10 mmol), malonic acid (1.4 g, 13 mmol) and 10 ml ethyl acetate with pyridine (0.5 ml, 0.5 g, 6 mmol) in a round-bottom flask and evaporated the solvent under reduced pressure at 60° C.

Once the solvent is completely evaporated, heat the reaction to 90° C. for 90 minutes.

After the reaction add ethyl acetate and extract with 1M hydrochloric acid in three steps and keep the organic layers. Wash the combined organic layers with saturated sodium chloride solution and demi-water. Dry the organic layer with magnesium sulfate, after filtration the organic layer can be evaporated under reduced pressure for product with 85% purity and 64% yield.

1. Process of producing a furan building block, comprising the steps of:
a) providing a furfural compound selected from the group consisting of hydroxymethylfurfural (HMF), hydroxymethyfurfural esters and hydroxymethylfurfural ethers;
b) reacting the furfural compound with an activated methylene compound comprising at least one electron withdrawing group in the presence of a catalyst to form a Knoevenagel product comprising a substituted furan compound comprising one or more electron-withdrawing groups and one or more carbon-carbon double bonds; and
c) subjecting the Knoevenagel product obtained in step b) to a reaction to form the furan building block, the obtained furan building block comprising at least one intact carboxylic acid functionality selected from —COOH, —C(═O)—O—CH₃ and —C(═O)—O—CH₂CH₃, the reaction being one of the following:
c1) a hydrogenation reaction;
c2) a decarboxylation reaction; and
c3) a de-esterification reaction.

2. Process according to claim 1, wherein the furan building block has the structure of formula (Ia) or the structure of formula (Ib): 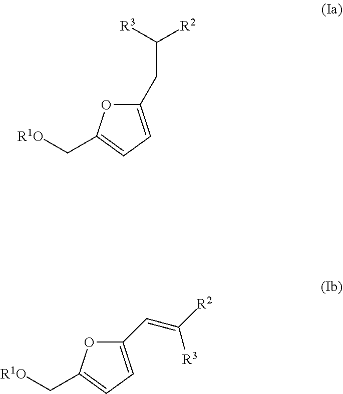
wherein R¹ represents hydrogen, —CH₃, —CH₂CH₃, or —C(═O)CH₃; R² represents —C≡N, —COOH, —C(═O)—O—CH₃ or —C(═O)—O—CH₂CH₃ and if R³ is present R³ represents —C≡N, —COOH, —C(═O)—O—CH₃ or —C(═O)—O—CH₂CH₃.

3. Process according to claim 1, wherein the activated methylene compound used in step b) is selected from the group consisting of malonic acid and malonic acid esters.

4. Process according to claim 1, wherein the furfural compound is hydroxymethylfurfural.

5. Process according to claim 1, wherein step b) comprises the steps of:
b1) combining the furfural compound, the activated methylene compound, the catalyst in a suitable solvent, to produce a liquid reaction mixture;
b2) keeping the liquid reaction mixture under conditions under which the Knoevenagel reaction proceeds, preferably at a temperature within the range of 40-120° C. and a pressure within the range of 1-5 bar.

6. Process according to claim 1, comprising the steps of:
a) providing a furfural compound selected from the group consisting of hydroxymethylfurfural (HMF), hydroxymethyfurfural esters and hydroxymethylfurfural ethers, in the form of an aqueous suspension, e.g. the crude or partially purified reaction mixture obtained by the acid-catalyzed dehydration of a hexose sugar;
b) reacting the furfural compound, in a medium comprising an organic solvent and water, with a malonic acid or an ester thereof in the presence of a catalyst to form a Knoevenagel product comprising a substituted furan compound comprising one or more electron-withdrawing groups and one or more carbon-carbon double bonds; followed by isolation or purification of the Knoevenagel product; and
c) placing the Knoevenagel product obtained in step b) in an aqueous solvent and subjecting it to a hydrogenation reaction, a decarboxylation reaction or a de-esterification reaction to form the furan compound.

7. Process according to claim 1, comprising the steps of:
a) providing a furfural compound selected from the group consisting of hydroxymethylfurfural (HMF), hydroxymethyfurfural esters and hydroxymethylfurfural ethers, in solid, e.g. in crystalline form;
b) reacting the furfural compound, in ethyl acetate, with a malonic acid ester in the presence of a catalyst to form a Knoevenagel product comprising a substituted furan compound comprising one or more electron-withdrawing groups and one or more carbon-carbon double bonds, followed by acid catalysed hydrolysis of the di-ester, whereby the Knoevenagel product precipitates from the ethyl acetate, following which the Knoevenagel product is separated from the liquid; and
c) placing the Knoevenagel product obtained in step b) in an aqueous solvent and subjecting it to a hydrogenation, a decarboxylation or a de-esterification reaction to form the furan compound.

8. Process according to claim 1, wherein the Knoevenagel product obtained in step b) is subjected to a hydrogenation reaction according step c1) with step c1) comprising the steps of
c1a) combining the Knoevenagel product and a catalyst in a suitable solvent, to produce a liquid reaction mixture;
c1b) contacting the liquid reaction mixture with hydrogen under conditions under which the hydrogenation proceeds, preferably at a temperature within the range of 20-120° C. and a pressure within the range of 1-10 bar.

9. Process according to claim 8, wherein the catalyst used in step c) is Raney nickel

10. Process according to claim 1, wherein the Knoevenagel product obtained in step b) is subjected to a decarboxylation reaction according step c2) with step c2) comprising the steps of subjecting the Knoevenagel product obtained in step b) to a reactive distillation.

11. Process according to claim 1, wherein the Knoevenagel product obtained in step b) is subjected to a decarboxylation reaction according step c3) with step c3) comprising the addition of KOH followed by the addition of NaHSO₃.

12. Process according to claim 1, further comprising the step d) of decarboxylating the furan compound as obtained in step c).

13. Process according to claim 12, further comprising the step e) of esterifying the furan building block as obtained in step d).

14. Process according to claim 12, wherein steps c)-d) or c)-e) are performed without isolation of the intermediates.

15. Process according to claim 12, wherein the furan building block has the structure of formula (II): 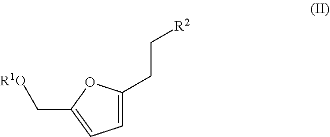
wherein R¹ represents hydrogen, —CH₃, —CH₂CH₃, or —C(═O)CH₃; and R² represents —C≡N, —COOH, —C(═O)—O—CH₃ or —C(═O)—O—CH₂CH₃.

16. Reaction product obtainable by the process of claim 1.
